(19) United States
(12) Patent Application Publication
(10) Pub. No.: US 2006/0200085 A1
(43) Pub. Date: Sep. 7, 2006

(54) Tissue transfer cannula and connectors

(76) Inventors: Philip Watts, Long Beach, CA (US); Brian F. Pockett, San Marcos, CA (US); David R. Koos, San Diego, CA (US)

Correspondence Address: THE LAW OFFICE OF JOHN A. GRIECCI, 703 PIER AVE., SUITE B #657, HERMOSA BEACH, CA, 90254, US

(21) Appl. No.: 11/356,318

(22) Filed: Feb. 15, 2006

Related U.S. Application Data
(60) Provisional application No. 60/653,396, filed on Feb. 15, 2005.
(60) Provisional application No. 60/696,309, filed on Jul. 1, 2005.
(60) Provisional application No. 60/720,189, filed on Sep. 23, 2005.

(51) Int. Cl. A61M 5/31

(52) U.S. Cl. 604/240

(57) ABSTRACT

A cannula including a shaft an outer layer and an inner layer, the inner layer being composed of a material that will cause the inner layer to be destroyed when autoclaved. A pre-applied layer of lubricant covers the inner and outer surfaces of the shaft. A slide is slidingly received within the shaft. An adaptor on the cannula includes a fitting forming a tapered bore sized to allow an outer end of a receptacle post to compressively contact the end of the shaft. An elastic sealing device is configured to be compressed between the fitting and the receptacle when the receptacle post is received within the tapered bore. This sealing device further seals the connection, protecting the tissue from contamination.

This application claims the benefit of U.S. provisional Application No. 60/653,396, filed Feb. 15, 2005, of U.S. provisional Application No. 60/696,309, filed Jul. 1, 2005, and of U.S. provisional Application No. 60/720,189, filed Sep. 23, 2005. Each of the aforementioned application(s) are incorporated herein by reference for all purposes.

This invention relates generally to tissue-handling medical devices, and more particularly to preferably disposable tissue-transfer devices such as cannula, syringes and related connectors.

### BACKGROUND

Typically, a syringe is provided with a receptacle configured to mate with comparable structure on a cannula. The receptacle is configured with a passageway to connect an interior cavity of a syringe barrel to a channel through the cannula. A common form of receptacle used to connect medical components, and place them in fluid communication, is a “LUER LOK”® receptacle. LUER LOK® receptacles come in many sizes, and many commonly used syringes are configured with these receptacles.

A LUER LOK® receptacle includes a cylindrical hub having an outer cylindrical surface that is smooth, and an inner cylindrical surface threaded with double lead female threads. The hub extends from an inner end affixed to, or unitary with, the syringe, to an outer end. The LUER LOK® receptacle further includes a tapered post extending concentrically through the hub. The tapered post extends from an inner end affixed to, or unitary with, the syringe, to an outer end extending beyond an outer end of the hub. The tapered post defines an outer surface with a circular cross-section, the diameter of which tapers uniformly from a larger size at the inner end of the tapered post to a smaller size at the outer end of the tapered post. The tapered post further defines an inner cylindrical surface, concentric with the outer surface and the hub. The inner surface forms a passageway through the tapered post. This passageway extends from an inner end that is in fluid communication with the interior of the syringe barrel, to an outer end that provides an opening to be placed in fluid communication with the cannula.

Existing adaptors configured to connect to a LUER LOK® receptacle are described throughout U.S. Pat. Nos. 5,002,538, and 6,569,118. Typically, these adaptors are characterized by having a fitting that receives the end of a medical device such as a cannula. The cannula end is provided with an opening that is to be placed in fluid communication with the inside of the syringe barrel. The fitting is configured to be screwed into the hub of the receptacle using the threads on the hub. The fitting is further configured to hold the cannula end opposing the tapered post of the receptacle, such that fluid can flow between the medical device and the passageway through the tapered post. The fitting defines a tapered bore for receiving the tapered post. The tapered bore of the fitting tapers down to a size smaller than the outer diameter of the tapered post at its outer end. As a result, when the fitting is screwed into the threads of the hub, the outer surface of the tapered post conforms to and squeezes against the tapered bore of the fitting, thus forming a seal to prevent the flow of fluid and gas. To provide for that seal to be tight, the fitting is designed to create the seal with the end of the shaft and the tapered post still a distance apart, thus providing a zone between the two in which the fluid flow path between the cannula and syringe is of a discontinuous size (e.g., diameter), and thus the fluid flow is disturbed rather than smooth.

Another common form of receptacle used to connect medical components, and place them in fluid communication, is a simple tapered end extending from the end of a larger syringe barrel. More particularly, the syringe body has a cylindrical barrel and a tapered post with a tapered outer surface, the post forming a passageway concentrically within the post, the passageway forming an opening at a distal end of the post. Such a receptacle is commonly used on 35 cc and 60 cc syringes. In typical form, an adaptor for such a receptacle includes a cylindrical portion to be conformingly received around the end of this syringe barrel with a friction fit, and an end portion, attached to the cylindrical portion, that is conformingly received over the tapered end of the syringe.

Using the above-described devices, sensitive withdrawn fluids (e.g., tissue to be therapeutically used such as stem cells derived from fat) can be disturbed by contact with the material of the shaft, by high levels of suction, and by discontinuous flow through the syringe/cannula interface. Such problems can be exacerbated by the tendency for tissue removal techniques to place the devices under high physical stress. Moreover, because of the importance in sanitary handling of such sensitive tissue, any errors in sanitizing of reusable equipment can cause significant contamination issues for the tissue to be therapeutically used.

Accordingly, there has existed a need for adaptors and related devices configured for use with commonly produced syringe receptacles, that provide for the handling of sensitive tissue with low levels of disturbance, such as by suction or discontinuous flow. Furthermore, there is a need for such adaptors and related devices to provide for good sealing characteristics and minimal risk of structural failure during use, as well as for minimizing potential contamination of removed tissue. Preferred embodiments of the present invention satisfy these and other needs, and provide further related advantages.

### SUMMARY

In various embodiments, the present invention solves some or all of the needs mentioned above, providing adaptors and related devices configured for use with syringe and other such receptacles, providing various advantages, such as in the handling of sensitive tissue with low levels of disturbance. Preferred variations of the adaptors and related devices provide for good sealing characteristics and minimal risk of structural failure during use, as well as for minimizing potential contamination of removed tissue.

In one aspect, the invention provides a cannula for connecting to a medical receptacle having a hub with an outside cylindrical surface and a concentric threaded inside cylindrical surface, and having a post with a tapered outer surface concentric within the hub, where the post forms a passageway concentrically within the post, and the passageway forms an opening at an outer end of the post. The cannula includes a shaft having an inner surface forming a channel within the shaft, and having an outer surface. The channel forms an opening at an end of the shaft. The shaft includes an outer layer and an inner layer, the inner layer being composed of a material that will cause the inner layer to be destroyed when autoclaved, advantageously preventing reuse of a cannula not intended for reuse.

The cannula further includes a layer of lubricant covering the inner surface of the shaft to provide for minimal viscous affect on passing tissue, and a layer of lubricant covering the outer surface of the shaft to provide for ease of use while the cannula is within a patient. The outer surface lubricant is pre-applied, and is covered by a packaging system configured to protect the layer of lubricant until the cannula is to be used. A slide slidingly received within the shaft provides additional protection to withdrawn tissue.

An adaptor is affixed to the shaft and configured for connecting the receptacle to the shaft. The adaptor includes a fitting attached to the end of the shaft, wherein the fitting includes an end portion forming a tapered bore concentric with the shaft channel, and opening into the shaft channel. The fitting end portion further forms an outer threaded surface configured for threadedly receiving the threaded inside cylindrical surface of the hub, and the tapered bore is configured to conformingly receive the tapered outer surface of the receptacle post as the fitting outer threaded surface threadedly receives the threaded inside cylindrical surface of the hub. The tapered bore is sized to allow the outer end of the post to compressively contact the end of the shaft, providing a first seal to the fluid passage, and minimizing discontinuities in the flow path of tissue passing through the cannula.

The fitting further includes an elastic sealing device configured to be compressed between the fitting and the receptacle when the receptacle post is received within the tapered bore. This sealing device further seals the connection, protecting the tissue from contamination.

The embodiments described below are provided with a number of novel concepts, each of which may separately be a basis of the invention. Among the objects of some of these inventions, is to create fluid tight seals; to provide for undisturbed, smooth fluid flow through an interface between a cannula and a syringe; to have fluid throwing through the cannula to be unaffected by the material of the cannula; to provide for a cannula to be cost effectively manufactured, and thus be disposable; to cause self-destruction of the cannula upon any attempt to sterilize the cannula for reuse, to provide uniform lubrication over the cannula exterior for the cannula to be safely lubricated for insertion into a patient; and to avoid exposing removed tissue to excessive disturbance due to the effects of suction.

Other features and advantages of the invention will become apparent from the following detailed description of the preferred embodiments, taken with the accompanying drawings, which illustrate, by way of example, the principles of the invention. The detailed description of particular preferred embodiments, as set out below to enable one to build and use an embodiment of the invention, are not intended to limit the enumerated claims, but rather, they are intended to serve as particular examples of the claimed invention.

### BRIEF DESCRIPTION OF THE DRAWINGS

FIG. 1 is a cross-sectional view of a first embodiment of a cannula having an adaptor, and a receptacle, under the invention.

FIG. 2 is a cross-sectional view of the adaptor and receptacle depicted in FIG. 1, attached to each other.

FIG. 3A is a cross-sectional view of a first variation of the adaptor depicted in FIG. 1.

FIG. 3B is a cross-sectional view of a second variation of the adaptor depicted in FIG. 1.

FIG. 3C is a cross-sectional view of a third variation of the adaptor depicted in FIG. 1.

FIG. 4A is a cross-sectional view of a second embodiment of an adaptor and a receptacle, under the invention.

FIG. 4B is a perspective view of the adaptor depicted in FIG. 4A.

FIG. 4C is a partial cross-sectional view of the adaptor and receptacle depicted in FIG. 4A.

FIG. 4D is a perspective view of a variation of the adaptor depicted in FIG. 1.

FIG. 5A is a partial cross-sectional view of a shaft of the cannula depicted in FIG. 1.

FIG. 5B is a cross-sectional view of a first variation of the cannula depicted in FIG. 1.

FIG. 5C is a cross-sectional view of a second variation of the cannula depicted in FIG. 1.

FIG. 6 is a cross-sectional view of a first embodiment of a connector having two adaptors, and two receptacles, under the invention.

FIG. 7 is a cross-sectional view of a second embodiment of a connector having two adaptors, under the invention.

FIG. 8 is a cross-sectional view of a third embodiment of a connector having two adaptors, under the invention.

FIG. 9 is a cross-sectional view of a fourth embodiment of a connector having two adaptors, under the invention.

### DETAILED DESCRIPTION OF THE PREFERRED EMBODIMENTS

The invention summarized above and defined by the enumerated claims may be better understood by referring to the following detailed description, which should be read with the accompanying drawings. This detailed description of particular preferred embodiments of the invention, set out below to enable one to build and use particular implementations of the invention, is not intended to limit the enumerated claims, but rather, it is intended to provide particular examples of them.

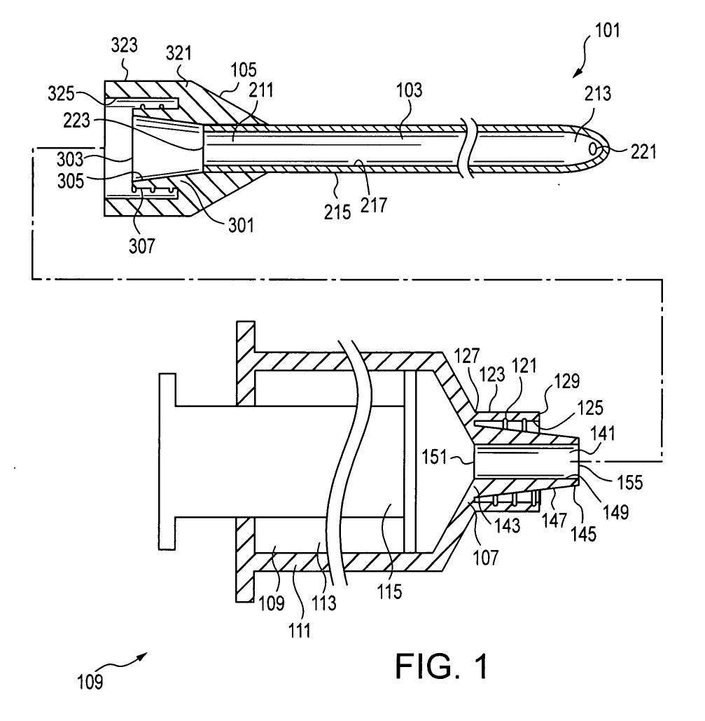

With reference to FIG. 1, a first embodiment of the invention is a cannula 101 that includes a shaft 103 and an adaptor 105. The adaptor is configured for use with a medical receptacle 107 of a syringe 109, such as a LUER LOK® receptacle. The syringe includes a barrel 111 that forms an interior cavity 113, and a piston 115.

The adaptor 105 may be made from a variety of materials, including metal, plastic, a graphite material, a ceramic material, or a carbon fiber material. Optionally, the adaptor may be composed of a material having material properties such that the adaptor is destroyed if the adaptor is heated in an autoclave at temperatures necessary to sterilize medical instruments. As a result, the cannula will be a single use, disposable unit that cannot be reused.

Preferably, the receptacle includes a cylindrical hub 121 having an outer cylindrical surface 123 that is smooth, and an inner cylindrical surface 125 threaded with double lead female threads. The hub extends from an inner end 127 affixed to, or unitary with, the syringe, to an outer end 129. The receptacle further includes a tapered post 141 extending concentrically through the hub. The post extends from an inner end 143 affixed to, or unitary with, the syringe, to an outer end 145 extending beyond an outer end of the hub. The post defines a tapered, conical outer surface 147 with a circular cross-section, the diameter of which tapers uniformly from a larger size at the inner end of the tapered post to a smaller size at the outer end of the tapered post. The tapered post further defines an inner cylindrical surface 149, concentric with the outer surface and the hub, which forms a passageway through the post. This passageway extends from an inner end 151 that is in fluid communication with the interior cavity 113 of the syringe, to an outer end 155 at the post outer end 145, providing an opening to be placed in fluid communication with the cannula. The post outer end forms a flat, ring-shaped end-surface parallel to the surface of the opening.

The shaft 103 is preferably composed of metal, such as is typically used for a cannula. Alternatively, the shaft may be made from a variety of materials, including plastic, a graphite material, a ceramic material, or a carbon fiber material. Optionally, the shaft may be composed of a material having material properties such that the shaft becomes destroyed if the shaft is heated in an autoclave at temperatures necessary to sterilize medical instruments. As a result, the cannula will be a single use, disposable unit that cannot be reused.

The shaft extends from a proximal end 211 to a distal end 213. The shaft has a cylindrical outer surface 215, and a concentric cylindrical inner surface 217 that forms a channel extending between the proximal and distal ends. The distal end of the cannula is closed, but is provided with an opening 221 configured for the cannula to be used for the removal or in injection of fluids. The proximal end of the shaft is open, and forms a flat, ring-shaped end-surface 223 conforming to the ring-shaped end-surface at the outer end 155 of the post 141.

The adaptor 105 is provided with a fitting 301 configured for attachment to the receptacle 107. The fitting is attached around the proximal end 211 of the shaft 103. The fitting includes an end portion 303 forming a tapered bore 305 that is concentric with the shaft channel, and that opens into the shaft channel. The fitting end portion further forms an outer threaded surface 307 configured for threadedly receiving the threaded inside cylindrical surface 125 of the hub. The tapered bore is configured to conformingly receive the tapered outer surface of the receptacle post when the fitting outer threaded surface 307 threadedly receives the threaded inside cylindrical surface 125 of the hub 121. Unlike existing adaptors, the tapered bore is sized such that the post may be inserted through the entire length of the tapered bore, allowing the longitudinal end at the outer end 145 of the post to press against the end of the shaft. Thus, the ring-shaped end-surface of the post outer end may come in to concentrically aligned opposing contact with the ring-shaped end-surface at the proximal end of the shaft.

The adaptor is further provided with an outer housing 321 concentrically surrounding the end portion 303 of the fitting 301. The housing defines a cylindrical outer surface 323 and a concentric cylindrical inner surface 325. The housing cylindrical inner surface approximately conforms to the outer surface 123 of the hub 121 when the adaptor is received on the receptacle, while allowing enough clearance for the housing to be slid over the hub. More particularly, the clearance does not provide a press fit over the hub, and does not form a seal to prevent leakage from the interface between the cannula and the attached syringe.

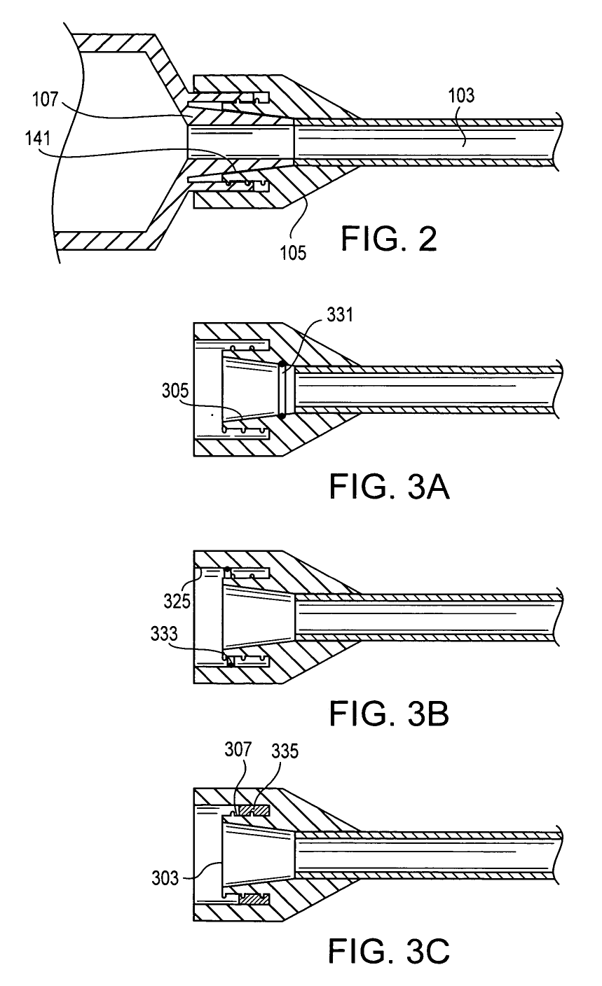

With reference to FIG. 2, the adaptor 105 may be attached to the receptacle 107. More particularly, with the post 141 and the shaft 103 concentrically aligned, the adaptor is slid over the receptacle such that the housing inner surface is received over the hub outer surface, and the post is received within the tapered bore of the fitting. The adaptor is then rotated with respect to the syringe such that the threads of the hub and the threads of the fitting threadedly receive each other, and such that the post advances further into the tapered bore of the fitting. The advance of the post into the tapered bore continues until the outer end of the tapered bore abuts the proximal end of the shaft. Additional torque on the adaptor frictionally locks the adaptor in place relative to the syringe, and presses the ring-shaped end-surface of the shaft and post into a tightly abutting relationship.

Preferably, the shaft channel has an inner diameter substantially the same as the inner diameter of the post passageway. Additionally, because the tapered surface of the post is conformingly received within the tapered surface of the fitting, the channel and the passageway are concentrically aligned such that fluid may flow between the channel and the passageway without any disturbance from significant variations or discontinuities along its flow path. Advantageously, this minimizes any damage that might occur to particularly sensitive fluids (e.g., fluids for stem cell cultures) that are being removed from a body.

In an alternative embodiment, the cannula channel may have an inner diameter over most of its length that is different than the diameter of the post passageway. For such a device, the proximal end of the shaft preferably includes a gradual taper such that the inner diameter of the channel at the proximal end of the shaft conforms to the inner diameter of the post passageway. Similar to a shaft having an equal sized channel as its post's passageway, this minimizes any damage that might occur to particularly sensitive fluids due to discontinuities in their flow path.

With reference to FIGS. 2 and 3A-3C, the adjoining end-surfaces of the post and the shaft of an attached cannula and syringe, form a first seal to limit the leaking of fluid from the cannula/syringe, or the leaking of air into the cannula/syringe. The interlaced threads of the fitting and hub provide a second seal to prevent leaking fluids or air.

Preferably an additional seal may be formed by using an elastic sealing device, such as an o-ring 331 received in a concentric groove around the diameter of the tapered bore 305 at a given longitudinal location within the tapered bore. Preferably, the o-ring is longitudinally close to the shaft to minimize its distance from the first seal. When the post is fully inserted into the tapered bore, it passes through this tapered-bore o-ring, compressing the o-ring to form an additional seal.

Alternatively, preferably an additional seal may be formed by using an elastic sealing device, such as an o-ring 333 received in a concentric groove around the diameter of the housing inner surface 325 at a given longitudinal location within the housing. When the housing is received over the hub, the hub passes through this housing o-ring, compressing the o-ring to form an additional seal.

As a second alternative, preferably an additional seal may be formed by using an elastic sealing device, such as a gasket 335, received around the outer threaded surface 307 of the fitting end portion 303. When the threaded outer surface of the fitting is screwed into the threaded inner surface of the hub, the outer end of the hub presses into the gasket, forming an additional seal around the circumference of the fitting end portion.

Optionally, any or all of the above seal mechanisms may be combined into a single adaptor to provide for a more redundant and complete seal system.

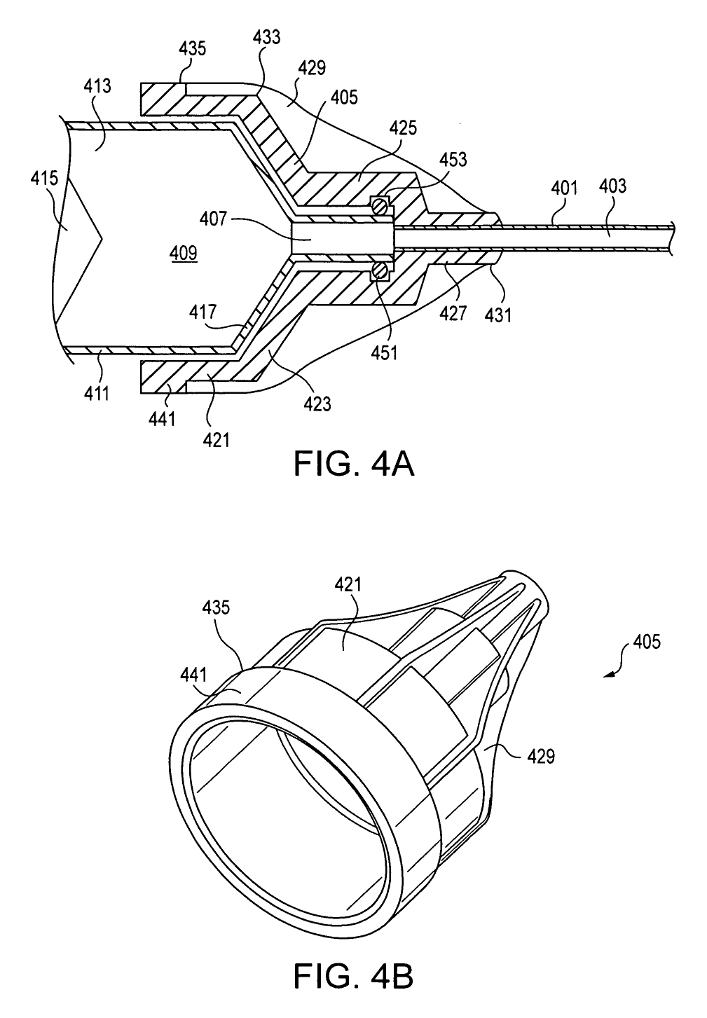

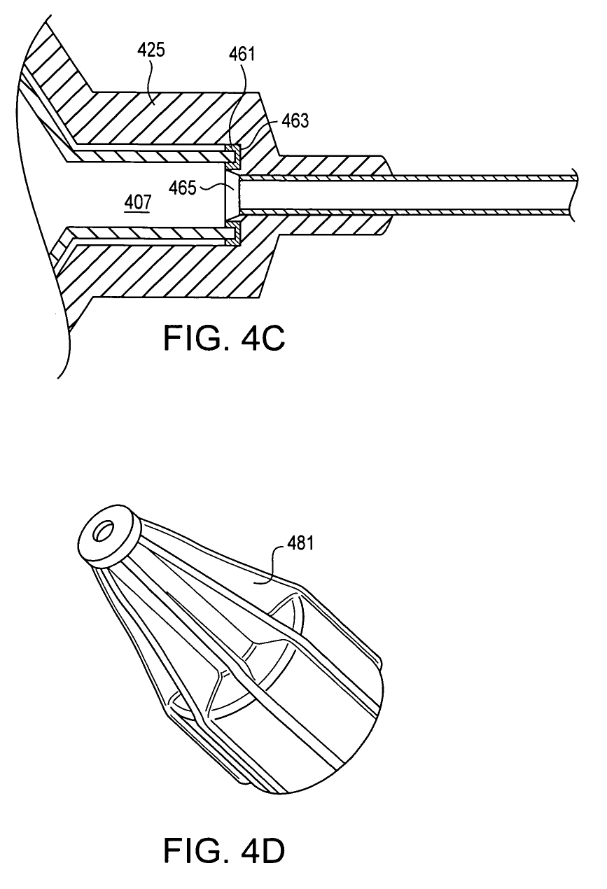

With reference to FIGS. 4A through 4B, a second embodiment of a cannula 401, includes a shaft 403 forming a channel, and an adaptor 405. The adaptor is configured for use with a simple receptacle post 407 of a syringe 409, such as are commonly used for 35 cc and 60 cc syringes. The syringe includes a barrel 411 that forms an interior cavity 413, and a piston 415 (i.e., a plunger). The barrel includes a cylindrical barrel portion, and an end portion including a conically tapered connecting portion 417 and the receptacle post 407, which is either not conically tapered or less conically tapered than the connecting portion. The connecting portion connects the receptacle post to the cylindrical barrel portion, such as is common for 35 cc and 60 cc syringes.

The adaptor 405 may be made from a variety of materials, such as a metal, a plastic, a graphite material, a ceramic material, or a carbon fiber material, and preferably it is a plastic material. Optionally, the adaptor may be composed of a material having material properties such that the adaptor is destroyed if the adaptor is heated in an autoclave at temperatures necessary to sterilize medical instruments. As a result, the cannula will be a single use, disposable unit that cannot be reused.

As with the previous embodiment, the shaft 403 is preferably composed of metal, such as is typically used for a cannula. Alternatively, the shaft may be made from a plastic, a graphite material, a ceramic material, a carbon fiber material or other such materials. Optionally, the shaft may be composed of a material having material properties such that the shaft becomes destroyed if the shaft is heated in an autoclave at temperatures necessary to sterilize medical instruments. As a result, the cannula will be a single use, disposable unit that cannot be reused. Previous discussions of the shaft are equally applicable to this embodiment.

The adaptor 405 includes a cylindrical portion 421 having a cylindrical inner surface to be conformingly received around an end of the cylindrical barrel portion with a friction fit, and an end portion, attached to the cylindrical portion, that is conformingly received over the end portion of the syringe. The adaptor end portion includes a conically tapered connecting portion 423 and a conical or conically tapered post portion 425 that is less tapered than the connecting portion. The post portion forms a bore concentric with the shaft channel, and opening into the shaft channel.

The bore is of the post portion is configured to conformingly receive the outer surface of the receptacle post. The adaptor cylindrical portion and end portion combine to form an internal surface conforming to the end of the barrel when the cylindrical portion is received over the end of the cylindrical barrel portion with a friction fit A distal end of the adaptor post portion (which forms the bore) is further configured with an extension portion 427 to extend beyond the end of the received receptacle post, providing a structural support connection to the shaft 403, placing the shaft in fluid communication with the receptacle, and sealing that fluid connection from exposure to the ambient environment.

In use, cannula adaptors often undergo significant torsional forces. The adaptor includes devices configured to maintain the structural integrity of the adaptor, to maintain the connection of the adaptor with the barrel, and to maintain the fluid communication between the barrel and the shaft without breaking the seal therebetween.

To these ends, the adaptor includes a plurality (and preferably eight or more) fins 429 (i.e., flanges) that preferably extend from a distal tip 431 of the post portion 425 at least to a distal end 433 of the cylindrical portion 421 (where it connects to the connecting portion 423), and more preferably near or at a proximal end 435 of the cylindrical portion 421. These fins provide support for the adaptor end portion, limiting its distortion under torsional forces, and therefore better maintaining the seal between the adaptor and the receptacle. These fins also provide support for the adaptor cylindrical portion, limiting localized bending under torsional forces, and therefore reducing the likelihood of strain related fractures.

The adaptor further includes a circumferential ring 441 distally extending from and around the proximal end 435 of the cylindrical portion 421. The cylindrical ring may be in the form of a thickened radially extending flange, and may receive distal ends of the fins. The ring preferably strengthens the proximal end of the cylindrical portion, thereby limiting strain related fractures. The circumferential ring preferably adjoins the ends of the fins, working in conjunction with them to strengthen the cylindrical portion in localized bending.

The post portion 425 forms a first seal to limit the leaking of fluid from the cannula/syringe, or the leaking of air into the cannula/syringe. The friction fit of the cylindrical portion over the barrel provides a second seal to prevent leaking fluids or air.

More particularly, the first seal may be formed by using an elastic sealing device, such as an o-ring 451 received in a concentric groove 453 around the inner diameter of the post portion 425 at a given longitudinal location. Preferably, the o-ring is longitudinally close to the shaft to minimize the distance between them. When the receptacle post 407 is fully inserted into the adaptor post portion, it passes through this o-ring, outwardly compressing the o-ring to form an additional seal.

With reference to FIG. 4C, as a second alternative, preferably an additional seal may be formed by using an elastic sealing device, such as a gasket 461, received at a distal end 463 of the internal surface of the adaptor post portion 425. When the receptacle post 407 is received into the adaptor post portion, the distal end of the receptacle post presses into the gasket, forming an additional seal.

Optionally, the gasket may be used in combination with a nozzle 465 located at the distal end of the tapered receptacle portion 425. The nozzle, which extends proximally from the distal end of the tapered receptacle portion, includes a cylindrical external surface configured to hold the gasket and receive the distal end of the receptacle, and a tapered conical inner surface configured to smoothly guide fluid flow. The nozzle serves both to assist in forming a seat for the receptacle against the gasket, and to guide fluid smoothly between the diameter of the shaft and the distal diameter of the receptacle. The nozzle also serves to prevent the gasket from extending out into the pathway of the fluid when the gasket is compressed by the distal end of the receptacle. The nozzle may also be used without the gasket.

Optionally, any or all of the above seal mechanisms may be combined into a single adaptor to provide for a more redundant and complete seal system.

Within the scope of the invention, the featured fins and/or ring may be used with other types of adaptor. For example, FIGS. 4D and 4E depict a variation of an adaptor of the first embodiment of the invention (above) adapted to have a similar set of fins 481, but no circumferential ring.

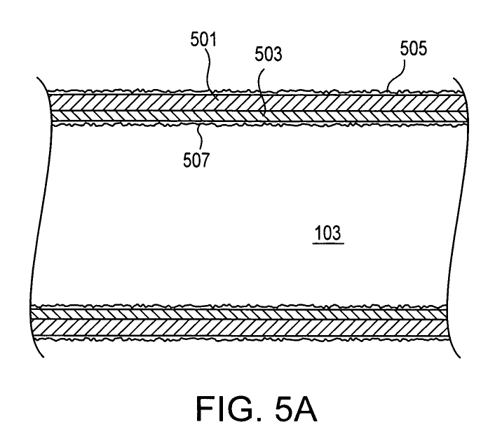

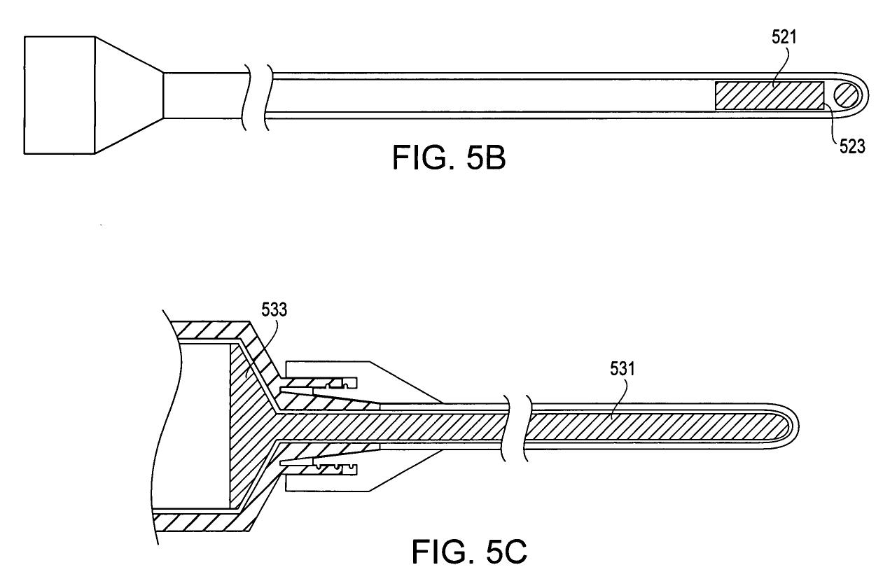

With reference to FIG. 5A the shaft 103 preferably includes an outer cylindrical layer 501 and an inner cylindrical liner 503. The outer cylindrical layer is preferably composed of metal, such as is typically used for a cannula. Alternatively, the outer cylindrical layer may be made from other materials, such as a plastic, a graphite material, a ceramic material, or a carbon fiber material. The inner cylindrical liner is preferably composed of a material having material properties such that the liner becomes destroyed if the shaft is heated in an autoclave at temperatures necessary to sterilize medical instruments. As a result, if the cannula is autoclaved (i.e., if it is placed in an autoclave and heated to a temperature appropriate for sterilizing medical instruments), the cannula becomes unusable. Thus, the cannula will be a single use, disposable unit that cannot be reused.

Preferably, the outer surface of the shaft is coted with a layer of external lubricant 505. This coating of external lubricant is preferably placed on the outer surface of the shaft during the manufacture of the cannula, and prior to the cannula being placed in sterile packaging for delivery to a final user. Preferably, the external lubricant is a polymer coating. Alternatively, the external lubricant may be of a type that is typically applied to a cannula by a physician immediately prior to using the cannula. As another alternative, a cannula user could apply the lubricant by hand as is presently known for traditional lubricants. Optionally, the external lubricant may include a disinfectant.

Preferably, the inner surface of the shaft, which is the an inner surface of the liner for embodiments including a liner, is coated with a layer of internal lubricant 507. The internal lubricant may be of the same type as the external lubricant, or may be of a different type. The internal lubricant is preferably placed on the inner surface of the shaft during manufacture of the cannula, and prior to the cannula being placed in sterile packaging for delivery to a final user. Preferably, the internal lubricant is a polymer, and forms a polymer coating on the inner surface of the shaft. Optionally, the internal lubricant may include a disinfectant, and/or cell culture materials. The layer of internal lubricant protects withdrawn fluids from contact with the internal surface of the shaft. This layer of internal lubricant also smooths the flow of fluid along the channel, further protecting the fluid from damage.

With reference to FIG. 5B, the cannula may be further provided with a slide 521. The slide is preferably a cylindrical plug conformingly received within the channel of the shaft. A distal end 523 of the slide may be planar (as depicted), or it may conform in shape to the distal end of the channel. The slide is preferably composed of a material aiding in the slide being able to slide freely along the length of the channel. This may be aided by using an internal lubricant as described above.

The slide is preferably positioned at the distal end of the cannula before use in removing a substance from a subject. The cannula is then inserted into the subject, with its opening adjoining the substance to be removed from the subject. The syringe piston is drawn from the syringe, causing a reduced pressure within the syringe to draw the slide toward the proximal end of the cannula. The slide begins to slide toward the proximal end of the cannula, and draws the substance to be removed in through the opening and into the channel. Because the substance is exposed primarily to the end of the slide, rather than to the low-pressure air within the proximal end of the channel, the substance is not exposed to the destructive effects of that low-pressure air. Advantageously, this cannula, with a slide, may be used with a standard syringe and piston. Measurement gradations on the syringe barrel or piston may be used to assess the amount of fluid withdrawn by the slide.

With reference to FIG. 5C, as an alternative to the slide being a cylindrical plug, the slide may be a shaft 531 extending substantially the entire length of the channel. The shaft is preferably attached directly to the piston of the syringe. Advantageously, this provides the user with precise control over the quantity of the substance withdrawn and the rate at which the substance is withdrawn from the user. Its use could lead to sharper variations in the speed with which the slide is withdrawn. Also, this full-length shaft slide requires either the use of an integral piston 533, or the use of a special attachment system for connecting the shaft to the piston.

The scope of the invention also includes tissue-transfer connectors usable to transfer tissue, such as fat containing stem cells, from one device to another while maintaining a sterile environment. The tissue-transfer connectors will typically comprise a first adaptor, a second adaptor, and a body forming a connecting passageway between the first and second adaptors.

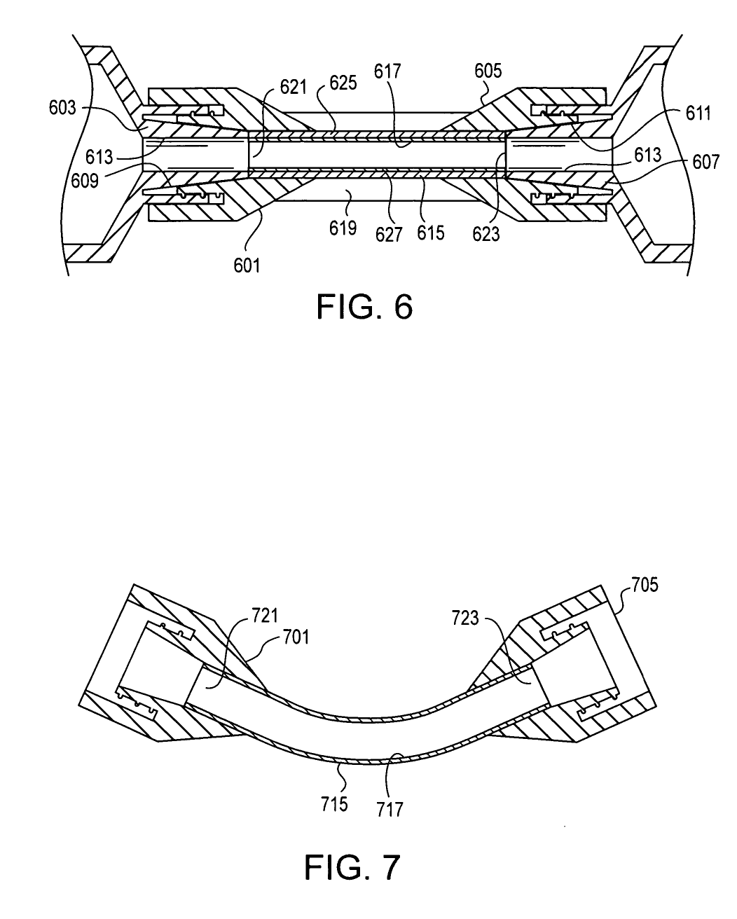

With reference to FIG. 6, a first embodiment of a tissue transfer connector includes a first adaptor 601 configured to mate with a first receptacle 603 such as a LUER LOK® connector, under the present invention (i.e., as is described above with reference to FIGS. 1-3), and a second adaptor 605 that is configured to mate with a second receptacle 607 such as a LUER LOK® connector, under the present invention. The first receptacle and second receptacle have a first post 609 and a second post 611, respectively, each post forming a passageway 613 for tissue passage. In this embodiment, the first and second receptacles are of the same size. A body 615 defines an inner surface 617 that forms a preferably 2.1 mm channel that places the first and second adaptors in fluid communication. The body is rigid, and is supported by stiff fins 619 extending from a first end 621 of the body that connects to the first adaptor, to a second end 623 of the body that connects to the second adaptor along an outer surface 625 of the body.

Similar to the cannula depicted in FIG. 2, preferably the body channel has a circular cross-section, and the body inner surface 617 has an inner diameter that is constant, and is substantially the same as the inner diameter of the post passageways of the first and second receptacles, on which the first and second adaptors are configured to be received. Similar to the discussion above, the adaptors are configured such that when the tapered surfaces of the receptacle posts are conformingly received within the tapered surfaces of the adaptor fittings, the body channel is concentrically aligned to the two receptacle passageways such that tissue may flow from the first receptacle passageway to the second receptacle passageway, via the channel, without any disturbance by significant variations or discontinuities along its flow path (e.g., without being affected by rapid variation in the channel cross-section).

In a variation of this embodiment, the tissue-transfer connector channel may have an inner diameter over most of its length that is different (e.g., larger to reduce flow resistance, or smaller to reduce residual tissue that could be left in the tissue-transfer connector) than the diameter of the post passageways of the two adaptors. For such a tissue-transfer connector, the body preferably includes a gradual tapering of the channel inner diameter such that the inner diameter of the channel at its two ends conforms to the inner diameters of the post passageways of the two adaptors. Similar to a tissue-transfer connector having a constant diameter channel, this minimizes any damage that might occur to particularly sensitive fluids due to discontinuities along their flow path.

In another variation of this embodiment, the second adaptor may be configured to fit a receptacle having a post-passageway inner diameter different than that for which the first adaptor is configured. For such a tissue-transfer connector, the body preferably includes a channel inner diameter that gradually tapers from a first size at the first end to a second size at the second end. The first size is substantially the same as the size of the post-passageway inner diameter for which the first adaptor is configured, and the second size is substantially the same as the size of the post-passageway inner diameter for which the second adaptor is configured.

As was previously described with respect to FIGS. 2 and 3A-3C, both adaptors are preferably configured such that the adjoining end-surfaces of the receptacle posts, and their respective ends of the tissue-transfer connector body, form seals to limit the leaking of fluid from the connections between the tissue-transfer connector and the two receptacles. The interlaced threads of each fitting and hub provide a second seal to prevent leaking fluids for air. Additional seals may be formed by using one or more elastic sealing devices, such as was described above.

With reference to FIG. 7, a second embodiment of a tissue-transfer connector is similar to the above-described first embodiment of a tissue-transfer connector, with the distinction that the body is flexible, and not typically provided with stiffeners. More particularly, this second embodiment of a tissue-transfer connector includes a first adaptor 701 and a second adaptor 705 similar to those described above with regard to the first embodiment of a tissue-transfer connector. A body 715 defines an inner surface 717 that forms a preferably 2.1 mm channel that places the first and second adaptors in fluid communication. The body forms a flexible tube, having a first end 721 that connects to the first adaptor, and a second end 723 that connects to the second adaptor.

Similar to the first embodiment of a tissue-transfer connector, preferably the body channel has an unflexed inner diameter that is constant and substantially the same as the inner diameter of the post passageways of the receptacles on which the first and second adaptors are configured to be received. Preferably, the body is circumferentially stiff enough to substantially maintain its channel circular shape and diameter when flexed. Likewise, the body is preferably longitudinally stiff enough to maintain channel integrity and insure that bends in the channel are gradual. Nevertheless, this embodiment is contemplated with variations similar to those described above for the first embodiment of the tissue-transfer connector, such that it is contemplated that the inner diameter of the channel could be made to vary along the length of the channel, and more preferably would vary gradually, such as if connecting receptacles of different sizes.

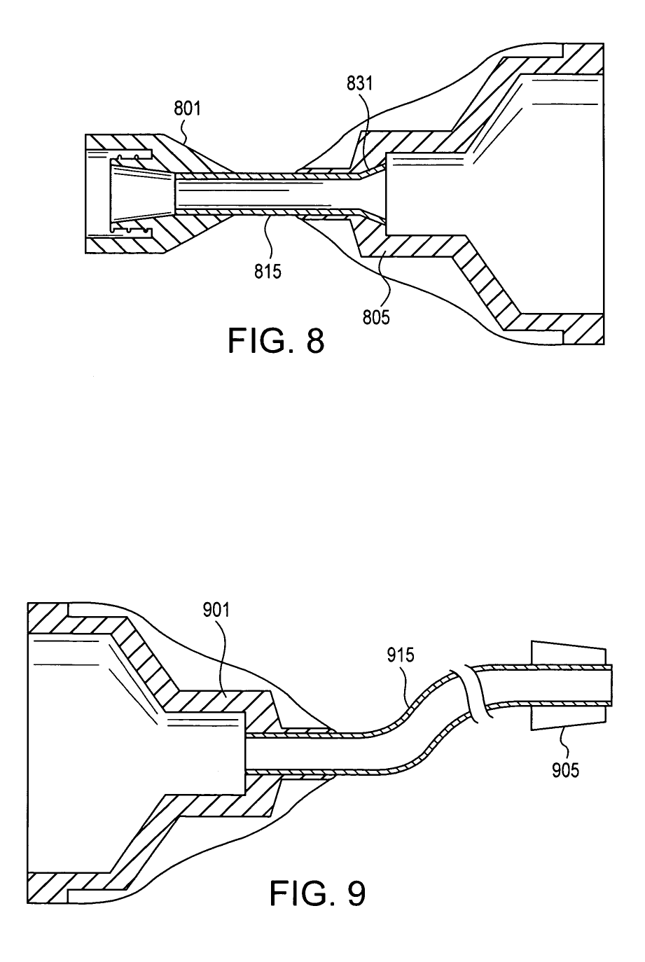

With reference to FIG. 8, a third embodiment of a tissue-transfer connector is similar to the above-described first embodiment of a tissue-transfer connector, with the distinction that the second adaptor is of type different than that of the first adaptor. More particularly, the third embodiment includes a first adaptor 801 similar to those described above with regard to the first embodiment of a tissue-transfer connector, and a second adaptor 805 configured to fit a different device, such as a 35 cc or 60 cc hub, as described above. As described with respect to the first embodiment of a tissue-transfer connector, a body 815 forms a channel that places the first and second adaptors in fluid communication. Optionally, the second adaptor and/or the second end of the body form a nozzle 831 configured to gradually connect and funnel tissue between the body channel and the device that the second receptacle is configured to receive. The embodiments of a tissue-transfer connector under the present invention include all 6 possible combinations of any given variation of the three adaptors described above (i.e., the adaptor for the Luer Lok®, the 35 cc adaptor, and the 60 cc adaptor), as well as the combinations of one of these three with other, industry standard adaptors (e.g., the present 60 cc adaptor with an industry standard Luer Lok® adaptor).

With reference to FIG. 9, other embodiments of a tissue-transfer connector may include a flexible body, as was described with respect to the second embodiment of the tissue-transfer connector. For example, FIG. 9 depicts a 60 cc hub receptacle adaptor 901 connected to a vacuum pump receptacle adaptor 905 via a flexible body 915.

With reference again to FIG. 6, similar to the cannula described above, all the various embodiments of a tissue-transfer connector under the present invention may further include a layer of internal lubricant 627 along the inner surface of the body (i.e., along the channel wall). The internal lubricant may be of the types described above for the internal lubricant of a cannula. The internal lubricant is preferably placed on the inner surface of the shaft during manufacture of the tissue-transfer connector, and prior to the tissue-transfer connector being placed in sterile packaging for delivery to a final user. Preferably, the internal lubricant is a polymer, and forms a polymer coating on the inner surface of the body. Optionally, the internal lubricant may include a disinfectant, and/or cell culture materials. The layer of internal lubricant protects transferred fluids from contact with the internal surface of the body. This layer of internal lubricant also smooths the flow of fluid along the channel, further protecting the fluid from damage.

As described above, with respect to cannula, the body of the tissue transfer connector preferably includes an outer layer and an inner layer, the inner layer being composed of a material that will cause the inner layer to be destroyed when autoclaved.

It is to be understood that the invention comprises related apparatus and methods for producing cannula and cannula-syringe systems, as well as the apparatus and methods of use for the cannula itself. The above disclosed features can be combined in a wide variety of configurations within the anticipated scope of the invention.

While particular forms of the invention have been illustrated and described, it will be apparent that various modifications can be made without departing from the spirit and scope of the invention. Thus, although the invention has been described in detail with reference only to the preferred embodiments, those having ordinary skill in the art will appreciate that various modifications can be made without departing from the scope of the invention. Accordingly, the invention is not intended to be limited by the above discussion, and is defined with reference to the following claims.

What is claimed is:

1. An adaptor for connecting a medical receptacle to a body, the receptacle having a post with a tapered outer surface, the post forming a passageway concentrically within the post, the passageway forming an opening at an outer end of the post, and the body forming a channel within the body, the channel forming a concentric opening at an end of the body, comprising:
a fitting attached to the end of the body;
wherein the fitting includes an end portion forming a tapered bore concentric with the body channel, and opening into the body channel;
wherein the tapered bore is configured to conformingly receive the tapered outer surface of the receptacle post; and
wherein the tapered bore is sized to allow the outer end of the post to compressively contact the end of the body.

2. The adaptor of claim 1, wherein the fitting further includes an elastic sealing device configured to be compressed between the fitting and the receptacle when the receptacle post is received within the tapered bore.

3. The adaptor of claim 1, the receptacle having a hub with an outside cylindrical surface and a concentric threaded inside cylindrical surface, wherein the tapered outer surface of the post is concentric within the hub, wherein:
the fitting end portion further forms an outer threaded surface configured for threadedly receiving the threaded inside cylindrical surface of the hub; and
the tapered bore is configured to conformingly receive the tapered outer surface of the receptacle post as the fitting outer threaded surface threadedly receives the threaded inside cylindrical surface of the hub.

4. A cannula, comprising:
the adaptor of claim 3;
a shaft having an inner surface forming a channel within the shaft, and having an outer surface, the channel forming an opening at an end of the shaft, wherein the shaft includes an outer layer and an inner layer, the inner layer being composed of a material that will cause the cannula to become unusable as a cannula when autoclaved;
a layer of lubricant covering the inner surface of the shaft;
a slide slidingly received within the shaft; and
wherein the fitting further includes an elastic sealing device configured to be compressed between the fitting and the receptacle when the receptacle post is received within the tapered bore.

5. A medical tissue transfer device, comprising:
the adaptor of claim 1; and
a body attached to the adaptor, the body forming a channel within the body that is in fluid communication with the adaptor.

6. A tissue-transfer connector for connecting a first receptacle to a second receptacle, the first receptacle having a post with a tapered outer surface, the post forming a passageway concentrically within the post, the passageway forming an opening at an outer end of the post, comprising:
the tissue transfer device of claim 5, being configured for attachment to the first receptacle; and
a second adaptor configured for attachment to the second receptacle;
wherein the body channel places the first and second adaptors in fluid communication.

7. A medical tissue transfer device, comprising:
a body having an inner surface forming a channel within the body; and
a layer of lubricant covering the inner surface of the body.

8. The tissue transfer device of claim 7, wherein the lubricant contains cell culture materials.

9. A medical tissue transfer device, comprising:
a body having an inner surface forming a channel within the body;
wherein the body includes an outer portion and an inner layer, the inner layer being composed of a material that will cause the tissue transfer device to become unusable as a tissue transfer device when autoclaved.

10. A medical tissue transfer device, comprising:
a body having an inner surface forming a channel within the body, and having an outer surface;
a layer of lubricant covering the outer surface of the body; and
a packaging system configured to protect the layer of lubricant until the tissue transfer device is to be used.

11. A medical tissue transfer device, comprising:
a body having an inner surface forming a channel within the body; and
a slide slidingly received within the channel.

12. A tissue-transfer connector for connecting a first receptacle to a second receptacle, comprising:
the tissue transfer device of claim 7, 9, 10 or 11,
a first adaptor configured for attachment to the first receptacle such that they are placed in fluid communication;
a second adaptor configured for attachment to the second receptacle such that they are placed in fluid communication; and
wherein the tissue transfer device channel places the first and second adaptors in fluid communication.

13. The tissue-transfer connector of claim 12, wherein the first and second receptacles are of the same size and configuration.

14. The tissue-transfer connector of claim 12, wherein the body is rigid.

15. The tissue-transfer connector of claim 12, wherein the body is flexible.

16. An adaptor for connecting a syringe body to a shaft, the syringe body having a cylindrical barrel and a post with an outer surface, the post forming a passageway concentrically within the post, the passageway forming an opening at a distal end of the post, and the shaft forming a channel concentrically within the shaft, the channel forming an opening at an end of the shaft, comprising:
a fitting attached to the end of the shaft;
wherein the fitting includes an end portion forming a bore concentric with the shaft channel, and opening into the shaft channel, the bore being configured to conformingly receive the outer surface of the post;
wherein the fitting further includes a cylindrical portion configured to conformingly receive the cylindrical barrel; and
wherein a proximal end of the cylindrical portion includes a circumferential ring configured to strengthen the proximal end.

17. An adaptor for connecting a syringe body to a shaft, the syringe body having a cylindrical barrel and a post with an outer surface, the post forming a passageway concentrically within the post, the passageway forming an opening at a distal end of the post, and the shaft forming a channel concentrically within the shaft, the channel forming an opening at an end of the shaft, comprising:
a fitting attached to the end of the shaft;
wherein the fitting includes an end portion forming a bore concentric with the shaft channel, and opening into the shaft channel, the bore being configured to conformingly receive the outer surface of the post;
wherein the fitting further includes a cylindrical portion configured to conformingly receive the cylindrical barrel; and
wherein the fitting further includes a plurality of fins extending from a distal end of the end portion to a proximal end of the cylindrical portion.

18. An adaptor for connecting a syringe body to a shaft, the syringe body having a cylindrical barrel and a post with an outer surface, the post forming a passageway concentrically within the post, the passageway forming an opening at a distal end of the post, and the shaft forming a channel concentrically within the shaft, the channel forming an opening at an end of the shaft comprising:
a fitting attached to the end of the shaft;
wherein the fitting includes an end portion forming a bore concentric with the shaft channel, and opening into the shaft channel, the bore being configured to conformingly receive the outer surface of the post;
wherein the fitting further includes a cylindrical portion configured to conformingly receive the cylindrical barrel; and
wherein the fitting further includes an elastic sealing device within the bore.

19. An adaptor for connecting a syringe body to a shaft, the syringe body having a cylindrical barrel and a post with an outer surface, the post forming a passageway concentrically within the post, the passageway forming an opening at a distal end of the post, and the shaft forming a channel concentrically within the shaft, the channel forming an opening at an end of the shaft, comprising:
a fitting attached to the end of the shaft;
wherein the fitting includes an end portion forming a bore concentric with the shaft channel, and opening into the shaft channel, the bore being configured to conformingly receive the outer surface of the post;
wherein the fitting further includes a cylindrical portion configured to conformingly receive the cylindrical barrel; and
wherein the fitting further includes a nozzle within the bore.
